(19) United States
(12) Patent Application Publication
(10) Pub. No.: US 2015/0112301 A1
(43) Pub. Date: Apr. 23, 2015

(54) APPARATUS AND METHOD FOR ENDOLUMINAL STENT TRANSIT

(71) Applicant: The Johns Hopkins University, Baltimore, MD (US)

(72) Inventors: Alexander Lewis Coon, Baltimore, MD (US); Geoffrey Philip Colby, Baltimore, MD (US)

(21) Appl. No.: 14/400,287

(22) PCT Filed: May 10, 2013
(86) PCT No.: PCT/US13/40622

§ 371 Date: Nov. 10, 2014

Related U.S. Application Data
(60) Provisional application No. 61/645,137, filed on May 10, 2012.

(51) Int. Cl. A61M 25/09

(52) U.S. Cl. CPC A61M 25/0905; A61M 2025/09008; A61M 2025/09175
USPC 604/506; 604/164.13; 604/96.01

(57) ABSTRACT

An endoluminal stent transit device. The endoluminal stent transit device includes a wire having a distal end and a proximal end, and a guide element attached to the wire at the distal end, which is movable between a first position and a second position. The guide element in the first position is in a contracted configuration while located within a lumen of a catheter and has a first diameter smaller than the catheter, and in the second position is in an expanded configuration while located outside of the lumen of the catheter and has a second diameter larger than the catheter.

### BACKGROUND OF THE INVENTION

With a growing number of cerebral aneurysms treated with endovascular stent/vascular reconstruction device (VRD)-assisted coiling and flow diverting devices (e.g., Pipeline embolization device), there exists the need for safe and efficacious strategies designed at crossing these in situ devices while maintaining endoluminal position of the catheter and wire and without damaging the in situ device.

This is often difficult using common techniques of a standard wire and microcatheter system to traverse the previously deployed stent/VRD, especially if the parent vessel is tortuous.

There are a number of reasons why crossing an in situ stent while maintaining an endoluminal position is important, regardless of whether it is a traditional stent or a flow diverter, and regardless of the treatment offered. These include retreating a recurrent/residual aneurysm, for example via additional coils, an additional stent, or a flow diverter, and gaining access to the distal blood vessel for treatment of a completely separate pathology, for example a distal aneurysm, stroke, arteriovenous malformation, tumor, etc.

The techniques used to traverse an in situ stent/VRD can often lead to malposition of the wire and microcatheter (i.e. between the stent/VRD and the wall of the vessel or between the stent/VRD and the aneurysm sac in cases where there is a free floating segment of stent/VRD within the aneurysm).

Accordingly, there is a need in the art for a device that eliminates the drawbacks of the traditional wire and microcatheter.

### SUMMARY

According to one embodiment, an endoluminal stent transit device is disclosed. The endoluminal stent transit device can include a wire having a distal end and a proximal end, and a guide element attached to the wire at the distal end which is movable between a first position and a second position. The guide element in the first position can be in a contracted configuration while located within a lumen of a catheter and can have a first diameter smaller than the catheter, and in the second position can be in an expanded configuration while located outside of the lumen of the catheter and can have a second diameter larger than the catheter.

According to another embodiment, a method for transiting a catheter through a lumen of a stent is disclosed. The method can include positioning a catheter proximal to an in situ stent. The catheter can define a lumen, and an endoluminal stent transit device can be disposed within the lumen in a first contracted configuration. The method can further include advancing the endoluminal stent transit device out of the lumen of the catheter, expanding the endoluminal stent transit device into a second expanded configuration, and advancing the endoluminal stent transit device into the in situ stent and through a length of the stent. The catheter can then be advanced over the endoluminal stent transit device into a desired position, and the stent transit device can be contracted into the first contracted configuration.

### BRIEF DESCRIPTION OF THE DRAWINGS

The accompanying drawings provide visual representations which will be used to more fully describe the representative embodiments disclosed herein and can be used by those skilled in the art to better understand them and their inherent advantages. In these drawings, like reference numerals identify corresponding elements and:

FIG. 1 illustrates a perspective view of a first step according to the features of the present invention.

FIG. 2 illustrates a perspective view of a second step according to the features of the present invention.

FIG. 3 illustrates an exploded view of a third step according to the features of the present invention.

FIG. 4 illustrates a perspective view of a fourth step according to the features of the present invention.

FIG. 5 illustrates a perspective view of a fifth step according to the features of the present invention.

FIG. 6A illustrates a perspective view of a sixth step according to the features of the present invention.

FIG. 6B illustrates a perspective view of a second part of the six step according to the features of the present invention.

FIG. 7 illustrates a perspective view of a seventh and eighth according to the features of the present invention.

FIG. 8 illustrates a perspective view of an alternative embodiment according to the features of the present invention.

FIG. 9 illustrates a perspective view of another alternative embodiment according to the features of the present invention.

FIG. 10 illustrates a perspective view of another alternative embodiment according to the features of the present invention.

FIG. 11 illustrates a perspective view of another alternative embodiment according to the features of the present invention.

### DETAILED DESCRIPTION OF THE PREFERRED EMBODIMENTS

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

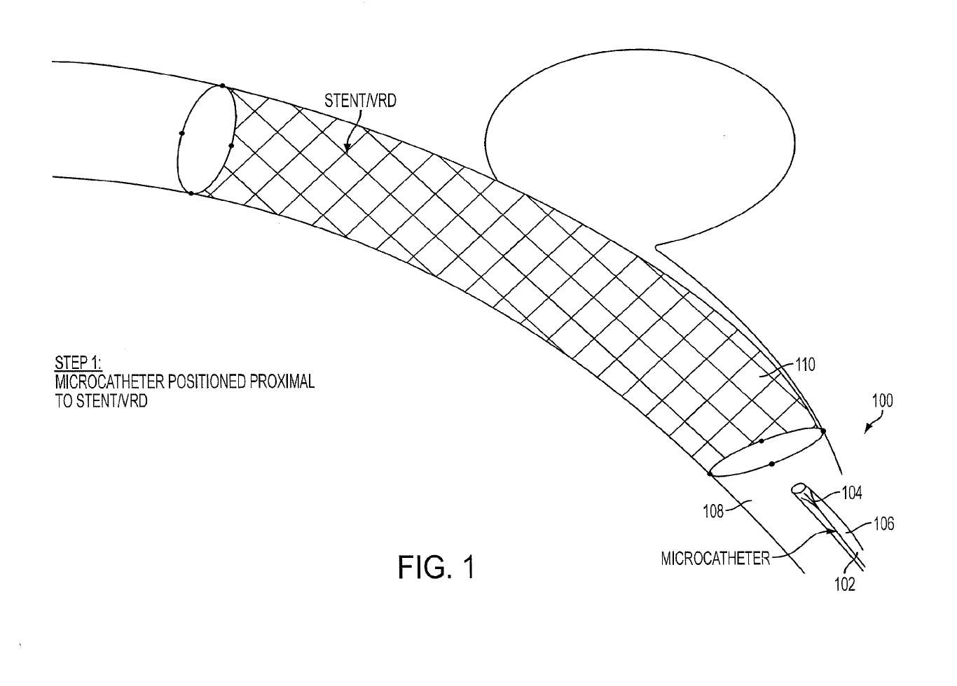

Generally, FIGS. 1-11 show embodiments of an endoluminal stent transit device (ESTD) and example techniques for using the same. All of the figures are drawn with a straight, non-tortuous segment of vessel just for the simplicity of illustration, but this device is also intended to be useful in other situations, for example tortuous vessels with sharp turns. FIG. 1 shows an ESTD 100 which can include a wire 102 with a distal end and a proximal end, and a guide element 104 attached to the distal end. Although the word wire is used herein, wire 102 is not limited to any particular configuration of wire, and can for example include a lumen within wire 102 so that it resembles a catheter or similar device. Guide element 104 can be movable between a contracted configuration and an expanded configuration.

FIG. 1 shows guide element 104 in a contracted configuration. When in the contracted configuration, guide element 104 and wire 102 can be located within a lumen of a catheter 106, which itself can be located in a lumen of a vessel 108, for example a blood vessel, and can be located proximate to or within an in situ stent 110 placed within vessel 108.

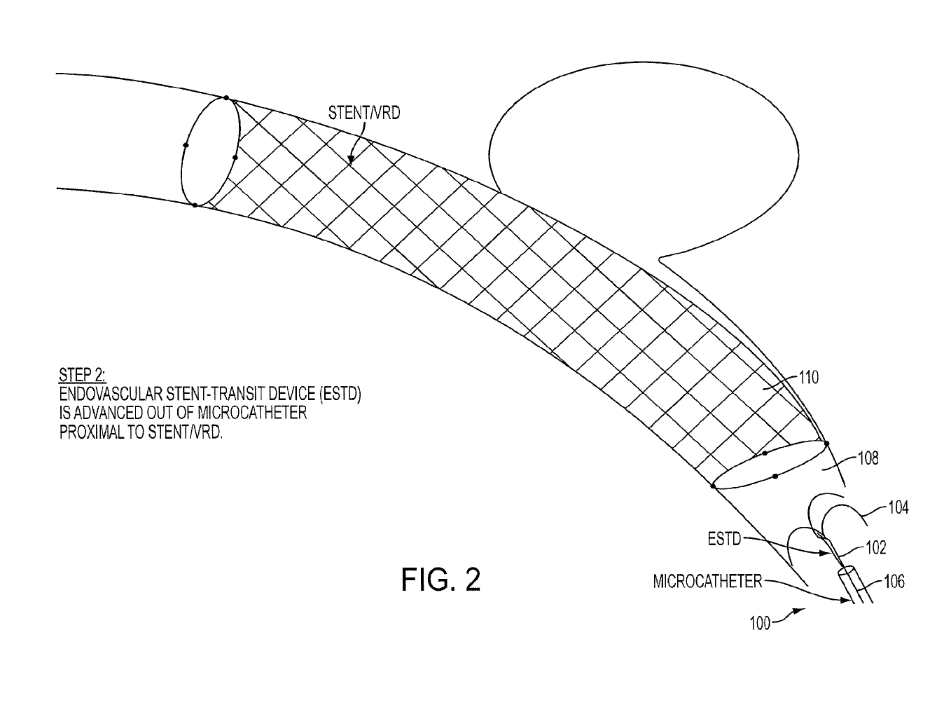

FIG. 2 shows ESTD 100 after it has been advanced out of catheter 106 so that guide element 104 and a portion of wire 102 are located outside of catheter 106. When this happens, guide element 104 can be expanded into an expanded configuration, with a diameter larger than catheter 106 but smaller than or equal to the diameters of vessel 108 and stent 110. This expansion can occur automatically, for example as a result of a release of tension caused by the exit of guide element 104 from catheter 106, or guide element 104 can expand as a result of a stimulus. While in an expanded configuration, portions of guide element 104 can come into contact with the walls of the lumen of vessel 108 or of stent 110. This contact can help to determine the radial position of ESTD 100 with respect to vessel 108 and stent 110, and can, for example, help to maintain the radial position of wire 102 as it transits vessel 108 and stent 110. The radial position of wire 102 can be, for example, substantially centered within vessel 108 and stent 110.

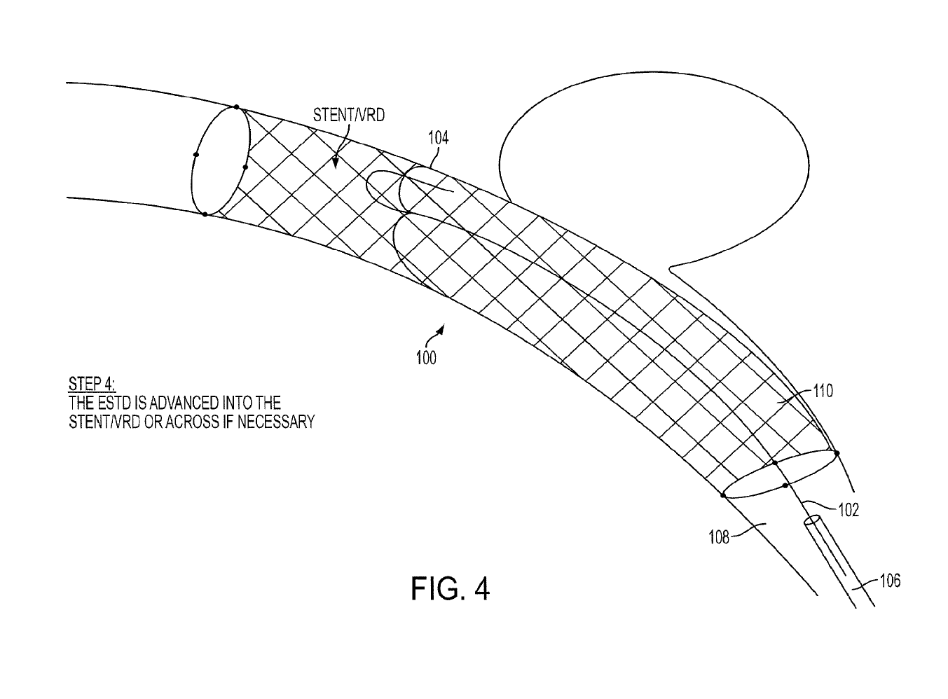

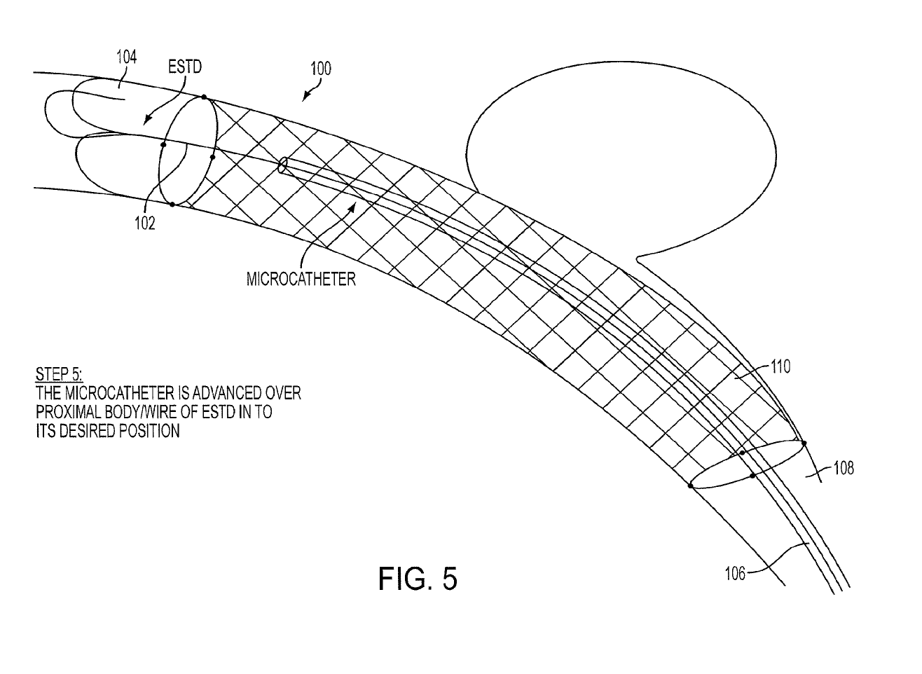

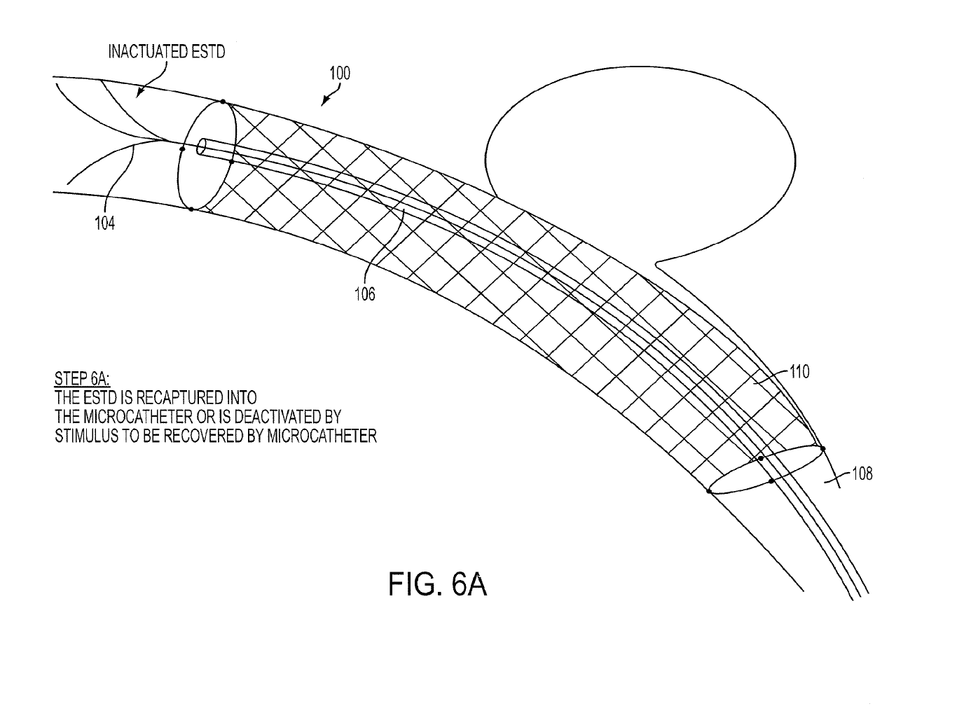

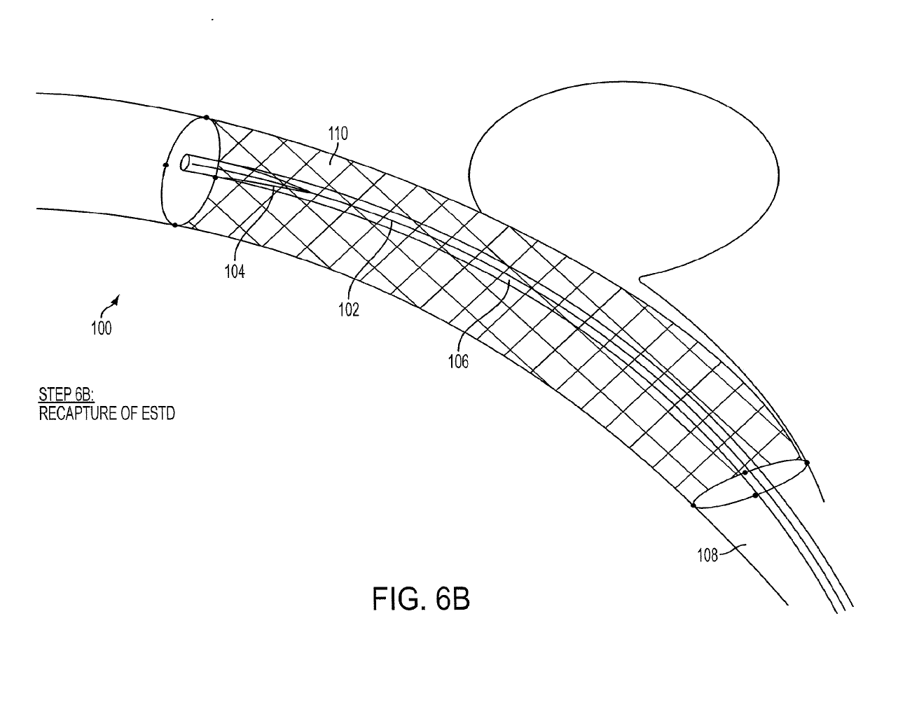

FIG. 4 shows ESTD 100 after it has been advanced into stent 110. FIG. 5 shows ESTD 100 after it has been advanced past stent 110. At any point before, during, or after the transit of ESTD 100 through stent 110, catheter 106 can be advanced along wire 102. In this way, guide 104 can assist in maintaining a radial position of catheter 106 with respect to vessel 108 and stent 110, for example a substantially centered position. FIGS. 6A and 6B show ESTD 100 as it is contracted into a contracted configuration and recovered or recaptured within catheter 106.

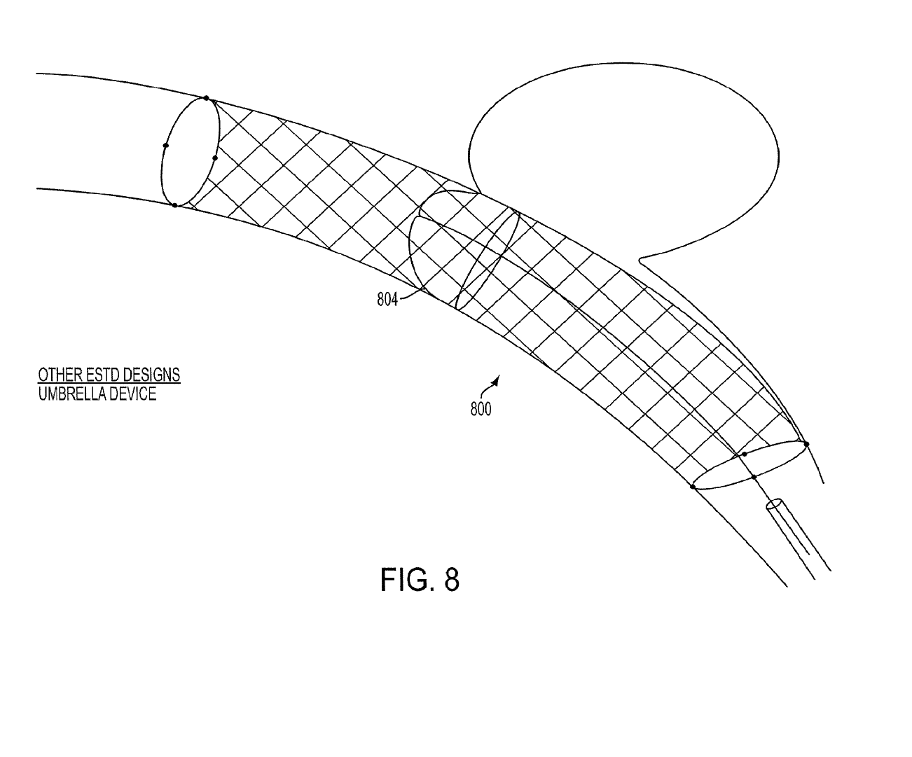

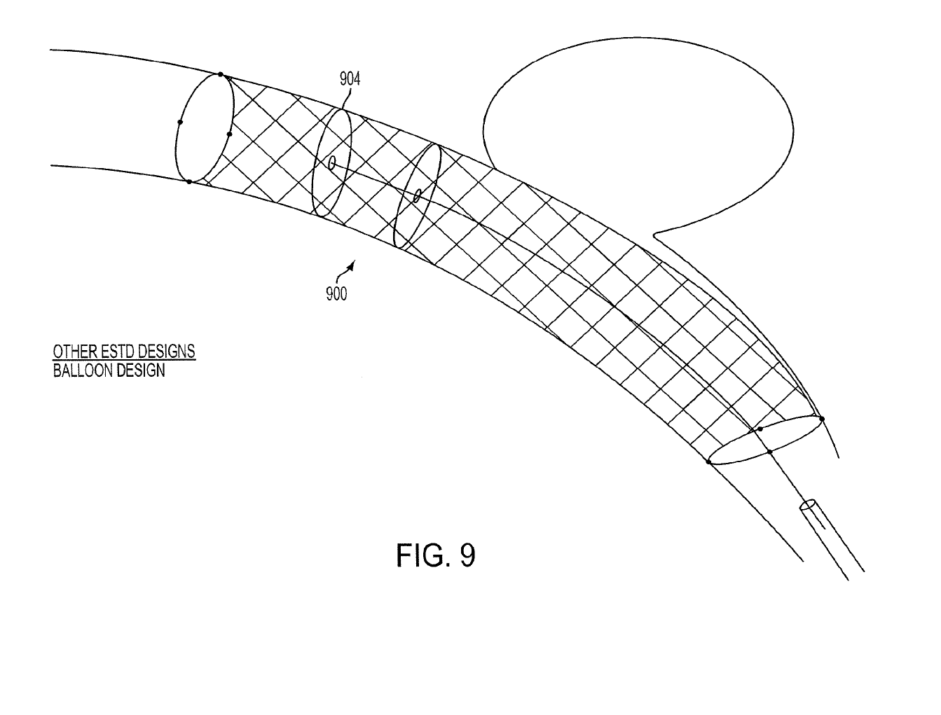

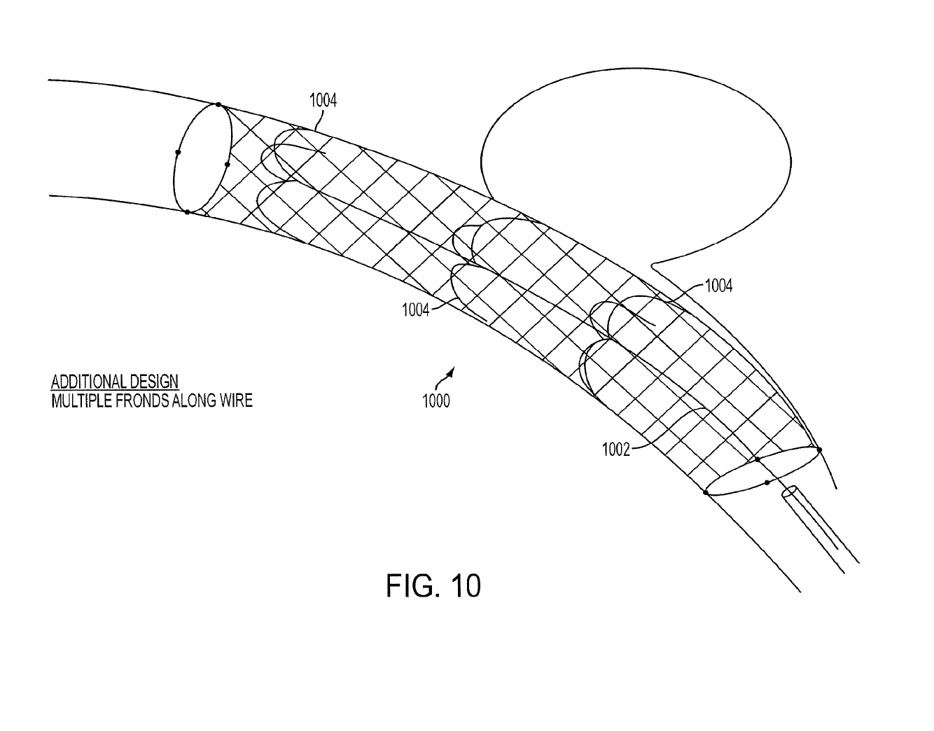

ESTDs can include guide elements of any any suitable shape with a diameter larger than catheter 106 while in an expanded configuration. FIG. 1 shows an example embodiment in which guide 104 has a plurality of prongs or fronds extending radially from wire 102. FIG. 8 shows an example embodiment of an ESTD 800 in which a guide element 804 is shaped as an umbrella. FIG. 9 shows an example embodiment of an ESTD 900 which includes guide element 904 shaped as a balloon which can be inflated in an expanded configuration. FIG. 10 shows an example embodiment of an ESTD 1000 which can include multiple guide elements 1004 located along wire 1002.

### EXAMPLES

The examples described below or intended for illustration only, and are not intended to be limiting in any way.

The present invention pertains to an endoluminal stent transit device (ESTD)/technique which is different from the current state of the art in that there are no devices or current techniques designed specifically for the safe selection of the inner lumen of an in situ stent and the transit of the wire across its length. The device described here can be designed to keep the wire tip towards the center of a vessel/stent/VRD lumen as it is advanced actively or passively into and across a stent (guaranteeing the wire is not between the wall of the vessel and the stent/VRD). This can be accomplished by a device tip that is atraumatic and can be passed bluntly. As used herein, the word “tip” can be refer to some embodiments of a guide element. Several examples of tip designs can include multiple wires arranged in a radial manner (as in the fronds of a palm tree as shown in FIG. 2), an umbrella shape (with the convex surface pointed anterior as shown in FIG. 8), or a balloon type tip that would float the wire into and through the stent/VRD, as shown in FIG. 9. In addition, there may be multiple fronds along a single wire, as shown, for example, in FIG. 10.

Conventional approaches use a wire or microwire which only have a single tip. This tip is typically blunted by being shaped or formed, but the tip is always solitary and therefore doesn't provide the protection and technical advantage of the described device.

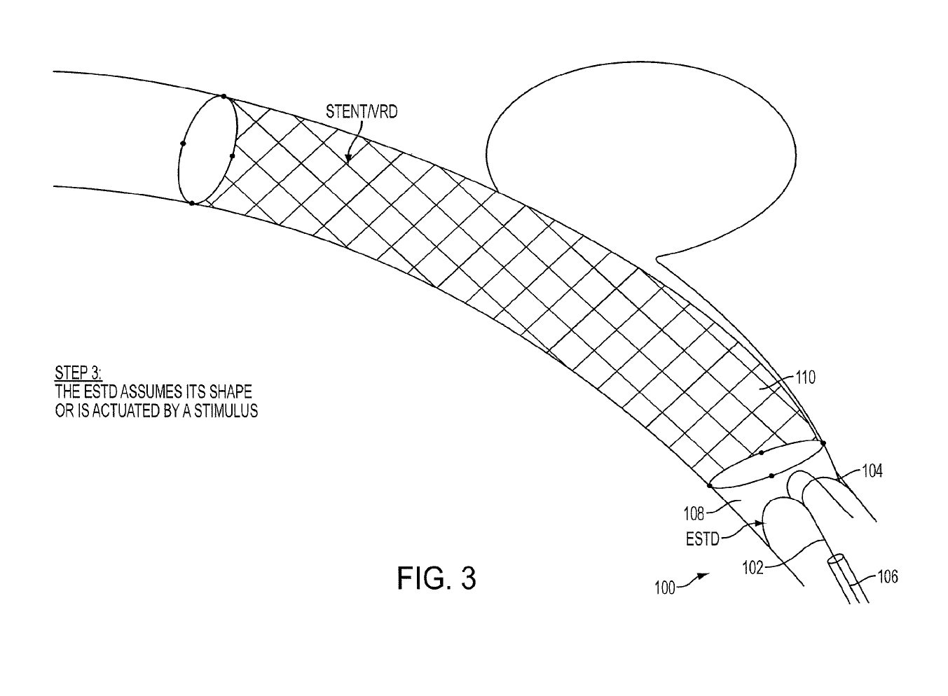

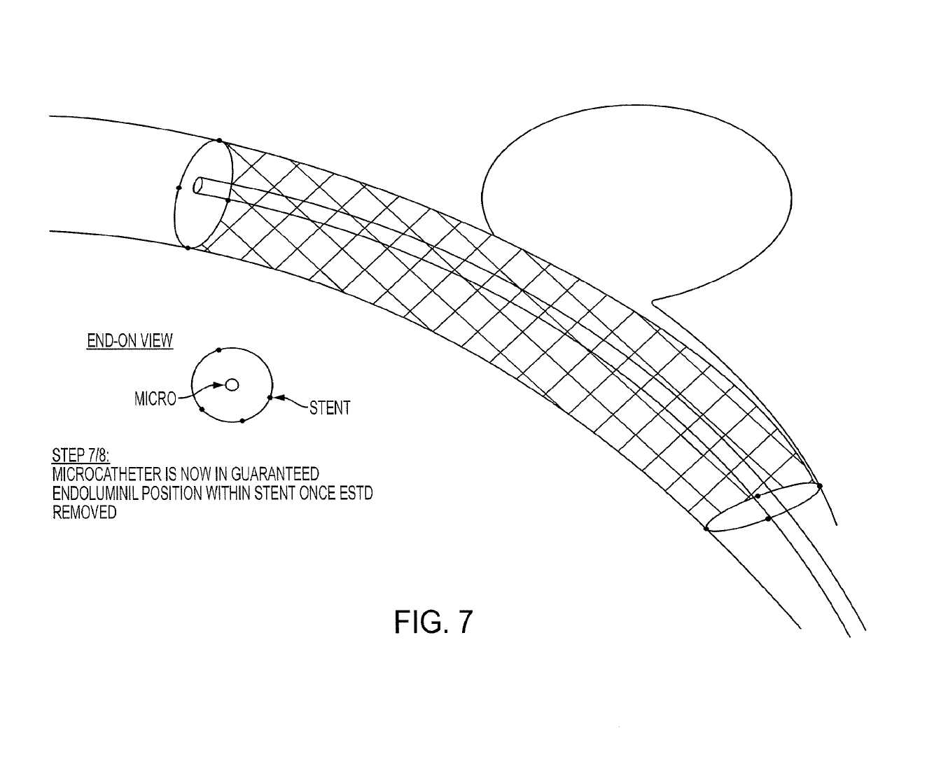

With reference to FIGS. 1-7, example steps for using the ESTD are illustrated. The discussion below refers to microcatheters, however methods of using the ETSD are not limited to microcatheters and can include any type of catheter or similar device as desired. With reference to FIG. 1, using standard over the wire microcatheter techniques, the microcatheter is positioned proximal to the vessel region of interest where the in situ stent/VRD is located. The endoluminal stent transit device (ESTD)/technique is advanced out of the microcatheter and into the vessel lumen proximal to the stent/VRD, as shown in FIG. 2. When the endoluminal stent transit device/technique exits the microcatheter tip, the front of the device either assumes its shape spontaneously or it is activated to reform via a stimulus, as shown in FIG. 3. Once properly shaped, the endoluminal stent transit device/technique is advanced into the in situ stent/VRD lumen and through the entire length of the stent if required, as shown in FIG. 4. Once access across the stent/VRD is achieved, the microcatheter is advanced into its desired position over the proximal body/wire of the device, as shown in FIG. 5. The device is then either deactivated by stimulus to form its original shape and then withdrawn into the microcatheter tip, or it is withdrawn into the tip and it spontaneously changes shape as it enters the microcatheter, as described in FIGS. 6A and 6B. The device is then withdrawn from the microcatheter and patient, as shown in FIG. 7. This has achieved safe and stable microcatheter position across the length of a stent/VRD that can be used for additional stent deployment and other techniques.

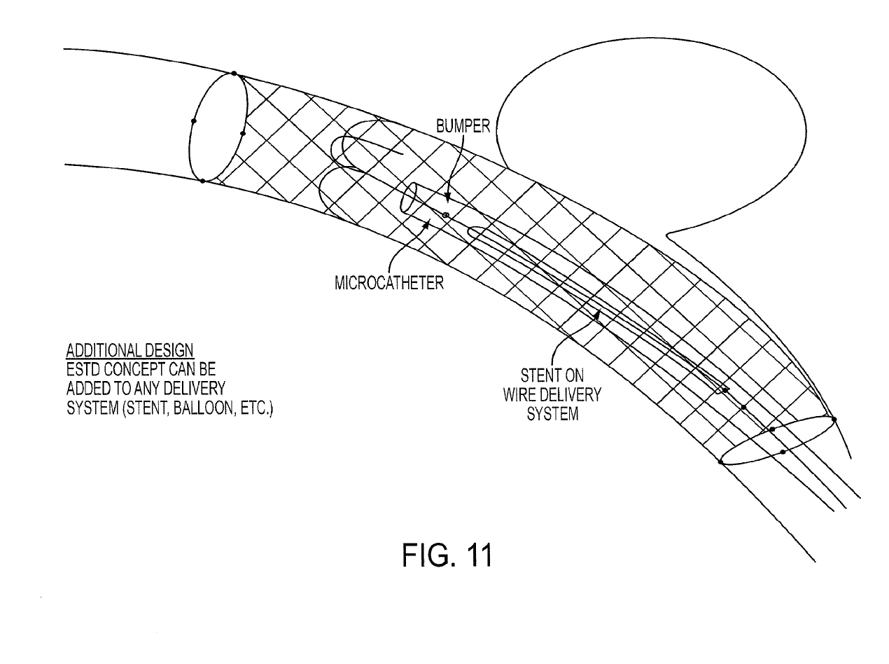

There can be numerous variations and modification the design of this device, all of which can achieve the same outcome of safe intraluminal transit of the device across an in situ stent/VRD, as shown for example, in FIGS. 8, 9, and 10. In addition, it should also be understood that the ESTD concept can be added to any delivery system (e.g., stent, balloon, etc.), as shown for example, in FIG. 11.

Although the present invention has been described in connection with preferred embodiments thereof, it will be appreciated by those skilled in the art that additions, deletions, modifications, and substitutions not specifically described may be made without departing from the spirit and scope of the invention as defined in the appended claims.

1. An endoluminal stent transit device, comprising
a wire having a distal end and a proximal end; and
a guide element attached to the wire at the distal end, the guide element movable between a first position and a second position,
wherein the guide element in the first position is in a contracted configuration while located within a lumen of a catheter and has a first diameter smaller than the catheter, and in the second position is in an expanded configuration while located outside of the lumen of the catheter and has a second diameter larger than the catheter.

2. The endoluminal stent transit device of claim 1, wherein the guide element while in the second position maintains a radial position of the wire within an inner lumen of an in situ stent while the wire transits the in situ stent.

3. The endoluminal stent transit device of claim 2, wherein the radial position is substantially centered within the inner lumen.

4. The endoluminal stent transit device of claim 1, wherein the guide element includes one or more fronds.

5. The endoluminal stent transit device of claim 1, wherein the guide element is shaped as an umbrella.

6. The endoluminal stent transit device of claim 1, wherein the guide element is shaped as a balloon.

7. A method for transiting a catheter through a lumen of a stent, comprising:
positioning a catheter proximal to an in situ stent, wherein the catheter defines a lumen, and an endoluminal stent transit device is disposed within the lumen in a first contracted configuration;
advancing the endoluminal stent transit device out of the lumen of the catheter;
expanding the endoluminal stent transit device into a second expanded configuration;
advancing the endoluminal stent transit device into the in situ stent and through a length of the stent;
advancing the catheter over the endoluminal stent transit device into a desired position; and
contracting the stent transit device into the first contracted configuration.
